(19) United States
(12) Patent Application Publication
(10) Pub. No.: US 2024/0078430 A1
(43) Pub. Date: Mar. 7, 2024

(54) DISENTANGLED WASSERSTEIN AUTOENCODER FOR PROTEIN ENGINEERING

(71) Applicant: NEC Laboratories America, Inc., Princeton, NJ (US)

(72) Inventors: Renqiang Min, Princeton, NJ (US); Hans Peter Graf, South Amboy, NJ (US); Tianxiao Li, New Haven, CT (US)

(21) Appl. No.: 18/449,748

(22) Filed: Aug. 15, 2023

Related U.S. Application Data
(60) Provisional application No. 63/403,894, filed on Sep. 6, 2022.

(51) Int. Cl. G06N 3/08

(52) U.S. Cl. CPC G06N 3/08

(57) ABSTRACT

A computer-implemented method for learning disentangled representations for T-cell receptors to improve immunotherapy is provided. The method includes optionally introducing a minimal number of mutations to a T-cell receptor (TCR) sequence to enable the TCR sequence to bind to a peptide, using a disentangled Wasserstein autoencoder to separate an embedding space of the TCR sequence into functional embeddings and structural embeddings, feeding the functional embeddings and the structural embeddings to a long short-term memory (LSTM) or transformer decoder, using an auxiliary classifier to predict a probability of a positive binding label from the functional embeddings and the peptide, and generating new TCR sequences with enhanced binding affinity for immunotherapy to target a particular virus or tumor.

### RELATED APPLICATION INFORMATION

This application claims priority to Provisional Application No. 63/403,894 filed on Sep. 6, 2022, the contents of which are incorporated herein by reference in their entirety.

### BACKGROUND

### Technical Field

The present invention relates to protein biophysics, and, more particularly, to learning disentangled representations for T-cell receptor designs for precise immunotherapy.

### Description of the Related Art

In protein biophysics, the separation between the functionally important residues (forming the active site or binding surface) and those that create the overall structure (the fold) is a well-established and fundamental concept. Identifying and modifying those functional sites is important for protein engineering but computationally non-trivial, and requires significant domain knowledge.

### SUMMARY

A method for learning disentangled representations for T-cell receptors to improve immunotherapy is presented. The method includes optionally introducing a minimal number of mutations to a T-cell receptor (TCR) sequence to enable the TCR sequence to bind to a peptide, using a disentangled Wasserstein autoencoder to separate an embedding space of the TCR sequence into functional embeddings and structural embeddings, feeding the functional embeddings and the structural embeddings to a long short-term memory (LSTM) or transformer decoder, using an auxiliary classifier to predict a probability of a positive binding label from the functional embeddings and the peptide, and generating new TCR sequences with enhanced binding affinity for immunotherapy to target a particular virus or tumor.

A non-transitory computer-readable storage medium comprising a computer-readable program for learning disentangled representations for T-cell receptors to improve immunotherapy is presented. The computer-readable program when executed on a computer causes the computer to perform the steps of optionally introducing a minimal number of mutations to a T-cell receptor (TCR) sequence to enable the TCR sequence to bind to a peptide, using a disentangled Wasserstein autoencoder to separate an embedding space of the TCR sequence into functional embeddings and structural embeddings, feeding the functional embeddings and the structural embeddings to a long short-term memory (LSTM) or transformer decoder, using an auxiliary classifier to predict a probability of a positive binding label from the functional embeddings and the peptide, and generating new TCR sequences with enhanced binding affinity for immunotherapy to target a particular virus or tumor.

A system for learning disentangled representations for T-cell receptors to improve immunotherapy is presented. The system includes a processor and a memory that stores a computer program, which, when executed by the processor, causes the processor to optionally introduce a minimal number of mutations to a T-cell receptor (TCR) sequence to enable the TCR sequence to bind to a peptide, use a disentangled Wasserstein autoencoder to separate an embedding space of the TCR sequence into functional embeddings and structural embeddings, feed the functional embeddings and the structural embeddings to a long short-term memory (LSTM) or transformer decoder, use an auxiliary classifier to predict a probability of a positive binding label from the functional embeddings and the peptide, and generate new TCR sequences with enhanced binding affinity for immunotherapy to target a particular virus or tumor.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of an exemplary system where the T-cell receptors (TCR) recognize antigenic peptides provided by the majority histocompatibility complex (MHC) with high specificity, in accordance with embodiments of the present invention;

FIG. 2 is a block/flow diagram of an exemplary disentangled autoencoder framework, in accordance with embodiments of the present invention;

FIG. 3 is a block/flow diagram of an exemplary method for sequence engineering, in accordance with embodiments of the present invention;

FIG. 4 is a block/flow diagram of an exemplary practical application for learning disentangled representations for T-cell receptors to achieve precision immunotherapy, in accordance with embodiments of the present invention;

FIG. 5 is a block/flow diagram of an exemplary processing system for employing a disentangled Wasserstein autoencoder for protein engineering, in accordance with embodiments of the present invention;

FIG. 6 is a block/flow diagram of an exemplary method for employing a disentangled Wasserstein autoencoder for protein engineering, in accordance with embodiments of the present invention; and

FIG. 7 is a block/flow diagram of an exemplary method for learning disentangled representations for T-cell receptors for precision immunotherapy, in accordance with embodiments of the present invention.

### DETAILED DESCRIPTION OF PREFERRED EMBODIMENTS

Decades of work in protein biology have shown the separation of the overall structure and the smaller “functional” site, such as the generic structure versus the active site in enzymes, and the characteristic immunoglobulin fold versus the antigen-binding complementarity-determining region (CDR) in immunoproteins. The latter usually defines the protein's key function, but cannot work on its own without the stabilizing effect of the former. This dichotomy is similar to the content-style separation in computer vision and natural language processing. For efficient protein engineering, it is often desired that the overall structure is preserved while only the functionally relevant sites are modified. Traditional methods for this task require significant domain knowledge and are usually limited to specific scenarios. Several recent studies make use of deep generative models or reinforcement learning to learn from large-scale data the implicit generation and editing policies to alter proteins. The exemplary methods tackle the problem by utilizing explicit functional features through disentangled representation learning (DRL), where the protein sequence is separately embedded into a “functional” embedding and a “structural” embedding. This approach results in a more interpretable latent space and enables more efficient conditional generation and property manipulation for protein engineering.

DRL has been applied to the separation of “style” and “content” of images, or static and dynamic parts of videos for tasks such as style transfer and conditional generation. Attaining the aforementioned disentangled embeddings in discrete sequences such as protein sequences, however, is challenging because the functional residues can vary greatly across different proteins. To this end, several recent works on discrete sequences such as natural languages use adversarial objectives to achieve disentangled embeddings. Other works improve the disentanglement with a mutual information (MI) upper bound on the embedding space of a variational autoencoder (VAE). However, this approach relies on a complicated implementation of multiple losses that are approximated through various neural networks, and involves finding a dedicated trade-off among them, making the model difficult to train.

To address these challenges, the exemplary methods propose a Wasserstein autoencoder (WAE) framework that achieves disentangled embeddings with a theoretical guarantee, using a simpler loss function. Also, WAE can be trained deterministically, avoiding several practical challenges of VAE in general, especially on sequences. The exemplary approach is proven to simultaneously maximize the mutual information (MI) between the data and the latent embedding space while minimizing the MI between the different parts of the embeddings, by minimizing the Wasserstein loss.

To demonstrate the effectiveness and utility of the exemplary method, the WAE is applied to the engineering of T-cell receptors (TCRs), which uses a similar structure fold as the immunoglobulin, one of the best-studied protein structures and a good example of separation of structure and functions. TCRs play an important role in the adaptive immune response by specifically binding to peptide antigens. Designing TCRs with higher affinity to the target peptide is thus of high interest in immunotherapy. Various data-driven methods have been proposed to enhance the accuracy of TCR binding prediction. However, there has been limited research on leveraging machine learning for TCR engineering.

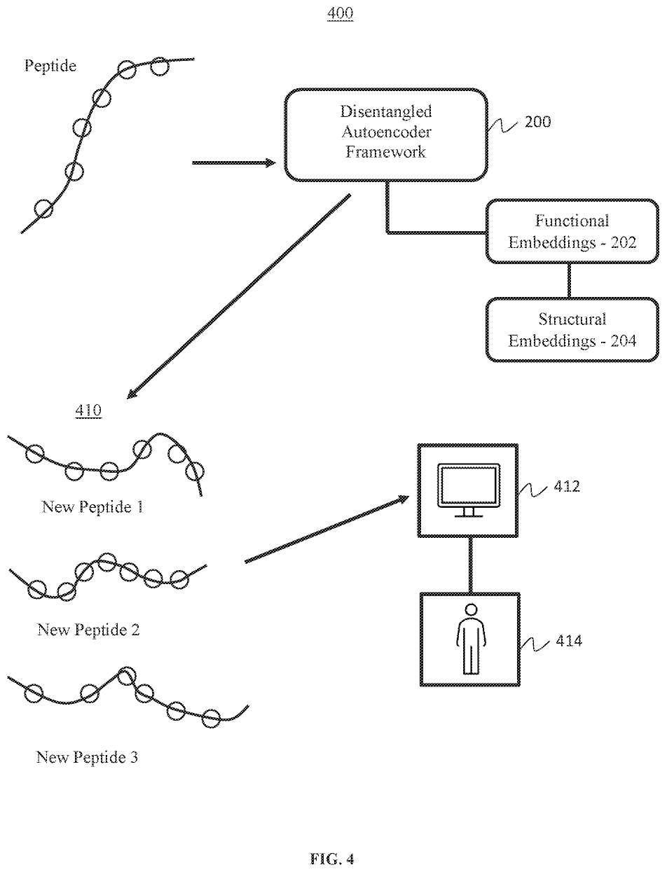

Using a large TCR-peptide binding dataset, the exemplary methods empirically demonstrate that the exemplary method successfully separates key patterns related to binding (“functional” embedding 202) from generic structural backbones (“structural” embedding 204) (FIG. 4). Furthermore, by modifying only the functional embedding, the exemplary approach is able to generate new TCRs with desired binding properties while preserving the structural backbone, needing only 10% of the running time needed by baseline models in comparison.

The contributions are as follows:

The exemplary methods are the first to formulate computational protein design as a style transfer problem and leverage disentangled embeddings for protein engineering, thus resulting in more interpretable and efficient conditional generation and property manipulation.

The exemplary methods introduce a disentangled Wasserstein autoencoder with an auxiliary classifier, which effectively isolates the function-related patterns from the rest with theoretical guarantees.

The exemplary methods show that by modifying only the functional embedding, the TCR sequences can be edited into desired properties while maintaining their backbones, running 10 times faster than baselines.

The exemplary embodiments define the problem of the TCR engineering task as follows. Given a TCR sequence and a peptide it could not bind to, a minimal number of mutations is optionally introduced to the TCR so that the TCR gains the ability to bind to the peptide. In the meantime, the modified TCR should remain a valid TCR, with no major changes in the structural backbone. Based on the assumption that only certain amino acids within the TCR should be responsible for peptide interactions, two kinds of patterns can be defined in the TCR sequence, that is, functional patterns and structural patterns. The former includes the amino acids that define the peptide binding property. TCRs that bind to the same peptide should have similar functional patterns. The latter refers to all other patterns which do not relate to the function but could affect the validity. The modeling is limited to the CDR3β region since it is the most active region for TCR binding. The TCR can further be referred to as a CDR3β region below.

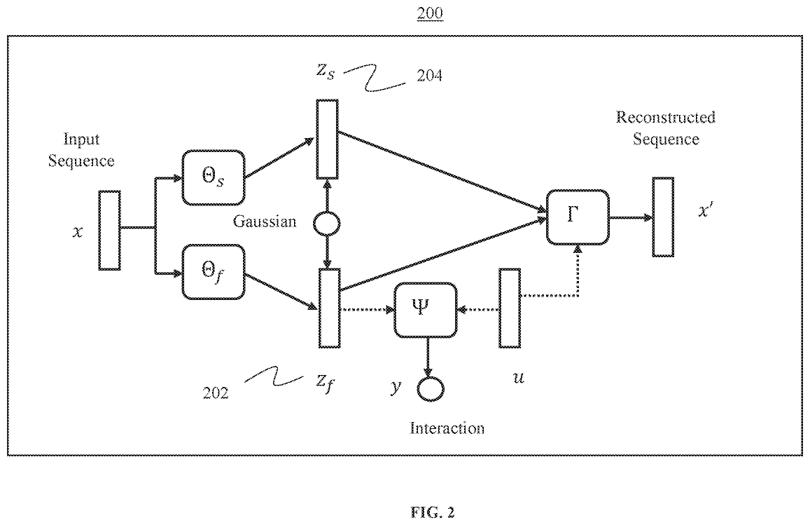

Regarding the disentangled Wasserstein autoencoder, the proposed framework, named TCR-dWAE, leverages a disentangled Wasserstein autoencoder 200 (FIG. 2) that learns embeddings corresponding to the functional and structural patterns. In this setting, the input data sample for the model is a triplet {x, u, y}, where x is the TCR sequence, u is the peptide sequence, and y is the binary label indicating the interaction.

In detail, given an input triplet {x, u, y}, the embedding space of x is separated into two parts, that is, z=concat(z_{f}, zₛ), where z_{f} is the functional embedding, and zₛ is the structural embedding.

Regarding the encoders and auxiliary classifier, the exemplary methods use two separate encoders for the embeddings, respectively:

zᵢ=Θᵢ(x),

where i∈{s, f} correspond to “structure” and “function.”

First, the functional embedding z_{f} is encoded by the functional encoder Θ_{f}(x). In order to make sure z_{f} carries information about binding to the given peptide u, an auxiliary classifier Ψ(z_{f}, u) is presented that takes z_{f} and the peptide u as input and predicts the probability of positive binding label q_{Ψ}(y|z_{f}, u),

ŷ=q_{Ψ}(Y=1|z_{f},u)=Ψ(z_{f},u).

The binding prediction loss is defined as binary cross entropy:

L_{f_cls}(ŷ,y)=−y log ŷ−(1−y)log(1−ŷ).

Second, the structural embedding zₛ is encoded by the structural encoder Θₛ(x). To enforce zₛ to include all the information other than the peptide binding-related patterns, a sequence reconstruction loss is leveraged.

Regarding the disentanglement of the embeddings, to attain disentanglement between z_{f} and zₛ, the exemplary methods introduce a Wasserstein autoencoder regularization term in the loss function, by minimizing the maximum mean discrepancy (MMD) between the distribution of the embeddings Z:Q_{z} where z=concat(z_{f}, zₛ) and an isotropic multivariate Gaussian prior Z₀:P_{Z} where P_{Z}=N (0, I_{d}):

L_{Wass}(Z)=MMD(P_{Z},Q_{Z}). (1)

The MMD is estimated as follows: given the embeddings {z₁, z₂, . . . , zₙ} of an input batch of size n, the exemplary methods randomly sample from the Gaussian prior {\tilde{z}₁, \tilde{z}₂, . . . , \tilde{z}ₙ} with the same sample size. The linear time unbiased estimator is then used to estimate the MMD:

$$MMD ( {P}_{Z} , {Q}_{Z} ) = \frac{1}{⌊ n / 2 ⌋} {\sum }_{i}^{⌊ n / 2 ⌋} h ( ( {z}_{2 i - 1} , {\tilde{z}}_{2 i - 1} ) , ( {z}_{2 i} , {\tilde{z}}_{2 i} ) ) ,$$

where h((zᵢ,\tilde{z}ᵢ),(zⱼ,\tilde{z}ⱼ)=k(zᵢ,zⱼ)+k(\tilde{z}ᵢ,\tilde{z}ⱼ)−k(zᵢ,\tilde{z}ⱼ)−k(zⱼ,\tilde{z}ᵢ) and k is the kernel function. Here a radial basis function (RBF) is used with σ=1 as the kernel.

By minimizing this loss, the joint distribution of the embeddings matches N (0, I_{d}), so that z_{f} and zₛ are independent.

Regarding the decoder and overall training objective, the decoder Γ takes z_{f}, zₛ and peptide u as input and reconstructs the original sequence as x′. The decoder also acts as a regularizer to enforce the structural embedding zₛ to include all the information other than the peptide binding-related patterns. The reconstruction loss is the mean position-wise binary cross entropy between x and x′:

$${x}^{′} = Γ ( concat ( {z}_{s} , {z}_{f} , u ) )$$

$${L}_{recon} ( x , {x}^{′} ) = {\sum }_{i}^{l} - {x}^{( i )} log ( {x}^{′ ( i )} ) - ( 1 - {x}^{( i )} ) log ( 1 - {x}^{′ ( i )} ) ,$$

where l is the length of the sequence and x⁽ⁱ⁾ is the probability distribution over the amino acids at the i-th position.

Combining all these losses, the final objective function is obtained, which then can be optimized through gradient descent in an end-to-end fashion:

L=L_{recon}+β₁L_{f_cls}+β₂L_{Wass}.

Regarding the disentanglement the guarantee, to show how the method can guarantee the disentangled embeddings, a novel perspective on the latent space of Wasserstein autoencoders is provided utilizing the variation of information.

A measurement of disentanglement is presented as follows:

D(Z_{f}, Zₛ;X|U)=VI(Zₛ;X|U)+VI(Z_{f};X|U)−VI(Z_{f};Zₛ|U),

where VI is the variation of information, VI(X;Y)=H(X)+H(Y)−2I(X;Y), which is a measure of independence between two random variables. For simplicity, the condition U (peptide) is omitted in the following parts.

This measurement reaches 0 when Z_{f} and Zₛ are totally independent, e.g., disentangled. It could further be simplified as:

VI(Zₛ;X)+VI(Z_{f};X)−VI(Z_{f};Zₛ)=2H(X)+2[I(Z_{f};Zₛ)−I(X;Zₛ)−I(X;Z_{f})].

It is noted that H(x) is a constant. Also, according to data processing inequality, as z_{f}→x→y forms a Markov chain, the exemplary methods have I(x;z_{f})≥I(y;z_{f}). Combining the results above, the upper bound of the disentanglement objective is given as:

I(Z_{f};Zₛ)−I(X;Zₛ)−I(X;Z_{f})≤I(Z_{f};Zₛ)−I(X;Zₛ)−I(Y;Z_{f}). (2)

Next, it is shown how the framework could minimize each part of the upper bound in (2).

For maximizing I(X; Zₛ), the following theorem is presented:

Given the encoder Q_{θ}(Z|X), decoder P_{γ}(X|Z), prior P(Z), and the data distribution P_{D}

D_{KL}(Q(Z)PP(Z))=E_{P}_{_{D}}[D_{KL}(Q_{θ}(Z|X)PP(Z))]−I(X; Z),

where Q(Z) is the marginal distribution of the encoder when X:P_{D} and Z:Q_{θ}(Z|X).

The theorem shows that by minimizing the KL divergence between the marginal Q(Z) and the prior P(Z), the exemplary methods jointly maximize the mutual information between the data X and the embedding Z, and minimize the KL divergence between Q_{θ}(Z|X) and the prior P(Z) . This also applies to the two separate parts of Z, Z_{f} and Zₛ. In practice, because the marginal cannot be measured directly, the aforementioned kernel MMD is minized instead.

As a result, there is no need for additional constraints on the information content of Zₛ because I(X; Z_{f}) is automatically maximized by the objective. It is noted that the exemplary methods also empirically verify that supervision on Zₛ does not improve the model performance.

For maximizing I(Y; Z_{f}) I(Y; Z_{f}) , a lower bound is given as follows:

I(Y;Z_{f})≥H(Y)+E_{p(Y,Z}_{_{f}}₎ log q_{Ψ}(Y|Z_{f},U),

where q_{Ψ}(Y|Z_{f}, U) is the predicted probability by the auxiliary classifier Ψ. Thus, maximizing the performance of classifier Ψ would maximize I(Y; Z_{f}).

For minimizing I(Z_{f}; Zₛ), minimization of the Wasserstein loss forces the distribution of the embedding space Z to approach an isotropic multivariate Gaussian prior P_{Z}=N(0, I_{d}), where all the dimensions are independent. Thus, the dimensions of Z will be independent, which also minimizes the mutual information between the two parts of the embedding, Z_{f} and Zₛ.

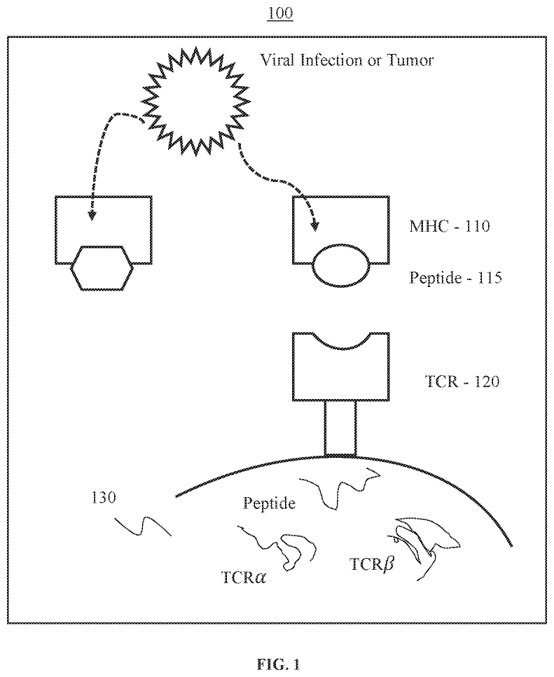

FIG. 1 is a block/flow diagram of an exemplary system 100 where the T-cell receptors (TCR) recognize antigenic peptides provided by the majority histocompatibility complex (MHC) with high specificity, in accordance with embodiments of the present invention.

The TCR 120 recognizes antigenic peptides 115 provided by the major histocompatibility complex (MHC) (110) with high specificity and the 3D structure 130 of the TCR-peptide-MHC binding interface (PDB: 5HHO) is provided.

FIG. 2 is a block/flow diagram of an exemplary disentangled autoencoder framework 200, in accordance with embodiments of the present invention.

The disentangled autoencoder framework 200 has an input x, that is, the CDR3β, and is embedded into a functional embedding z_{f} (202) and structural embedding zₛ (204).

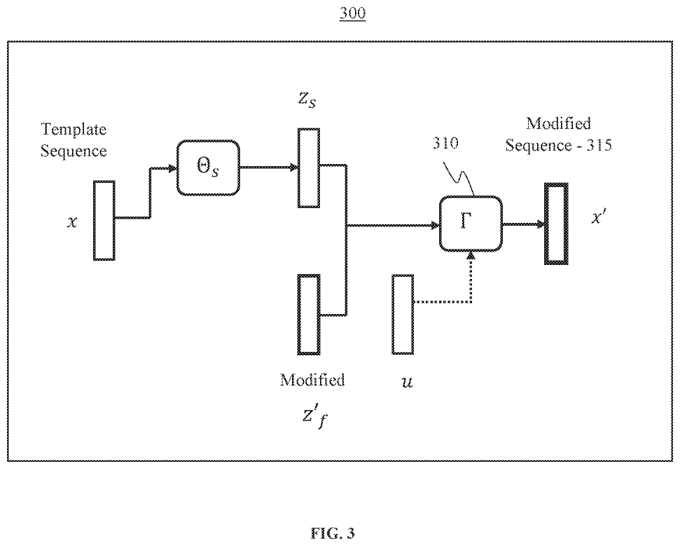

FIG. 3 is a block/flow diagram of an exemplary method 300 for sequence engineering, in accordance with embodiments of the present invention.

The method 300 for sequence engineering has an input x. The method 300 further has zₛ of the template sequence and a modified z_{f}, which represents the desired peptide binding property. These are fed to the decoder 310 to generate the engineered TCRs x′ (or modified sequence 315).

FIG. 4 is a block/flow diagram of an exemplary practical application for learning disentangled representations for T-cell receptors to achieve precision immunotherapy, in accordance with embodiments of the present invention.

In one practical example 400, a peptide is processed by the disentangled autoencoder framework 200 to separate functional embeddings 202 from the structural embeddings 204, to generate new peptides 410 to be displayed on a screen 412 and analyzed by a user 414.

The TCR-dWAE model 200 uses two transformer encoders for Θₛ, Θ_{f} and a long short-term memory (LSTM) recurrent neural network or transformer decoder for Γ. The auxiliary classifier Ψ is a 2-layer perceptron. Hyperparameters are selected through, e.g., a grid search. All results are averaged across, e.g., four random seeds.

As shown in FIG. 3, given any TCR sequence template x, the exemplary methods combine its original zₛ and a new functional embedding z′_{f} that is positive for the target peptide u (e.g., T(z′f, u)>0.5), which is then fed to the decoder 310 to generate a new TCR x′ (315) that could potentially bind to u while maintaining the backbone of x.

The exemplary methods obtain the positive z′_{f} in the following ways:

Random: z_{f} of a randomly selected positive TCR.

Best: the z_{f} that produces the highest classifier prediction.

Average: the average of z_{f}'s of all positive sequences. As a negative control, the exemplary methods also use z_{f} randomly sampled from a multivariate normal distribution, labeled null.

The exemplary methods use the following metrics to evaluate whether the engineered sequence x′ is a valid TCR sequence and binds to the given peptide, which is denoted as a validity score and a binding score, respectively.

The validity score rv evaluates whether the generated TCR follows similar generic patterns as naturally observed TCRs from TCRdb, an independent and much larger dataset. The exemplary methods train another autoencoder on TCRdb. If the generated sequence can be reconstructed successfully by the autoencoder and has a similar embedding pattern as the known TCRs, the exemplary methods consider it as a valid TCR following a similar distribution as the known ones. The exemplary methods also show that this metric separates true TCRs from other protein segments and random sequences.

For the binding score, the engineered sequence x′ and the peptide u are fed into a pre-trained ERGO classifier and binding probability rb=ERGO(x′, u) is calculated.

In general, TCR-dWAE-based methods generate more valid and positive sequences compared to other methods. One advantage of TCR-dWAE is that zₛ implicitly constrains the sequence backbone, ensuring validity. TCR-dWAE can perform sequence engineering in one pass, requiring 10× less time.

In conclusion, the exemplary methods propose an autoencoder model with disentangled embeddings where different sets of dimensions correspond to generic TCR sequence backbones and binding-related patterns, respectively. The disentangled embedding space improves interpretability of the model and enables optimization of TCR sequences conditioned on antigen binding properties. By modifying the binding-related parts of the embedding, TCR sequences with enhanced binding affinity can be generated while maintaining the backbone of the template TCR. The exemplary methods approach the TCR optimization task in a similar fashion as a style transfer problem in natural language processing, where the “style” (e.g., writing style, tone or sentiment) of a sentence is modified while the “content,” namely the general meaning, is maintained. This is based on the consideration that for a TCR, a user would like to modify a limited number of sites to enhance the binding affinity, while preserving the sequence backbone so that the optimized sequence is still a valid TCR. Different from some previous methods where mutations are iteratively added to the sequence, a style transfer model requires only one pass to generate sequences. Also, “style” or functional embedding, separated from the “content” or sequence backbone, could be used as a novel predictive feature for the binding affinity of the TCR, which would facilitate model interpretation and large-scale conditioned generation.

Therefore, the exemplary methods design a disentangled autoencoder that embeds the TCR sequence into a “functional” embedding and a “sequential” embedding, where the former includes information about the generic sequential context and the latter encodes patterns that are responsible for peptide recognition. Two auxiliary losses to ensure the functional embedding encodes the functional information, while being independent from the sequential embedding. The exemplary methods then modify the functional embedding for known non-binding TCRs given the peptide to generate new binding TCRs. The exemplary system can be used for generating TCRs for immunotherapy targeting a particular type of virus or tumor.

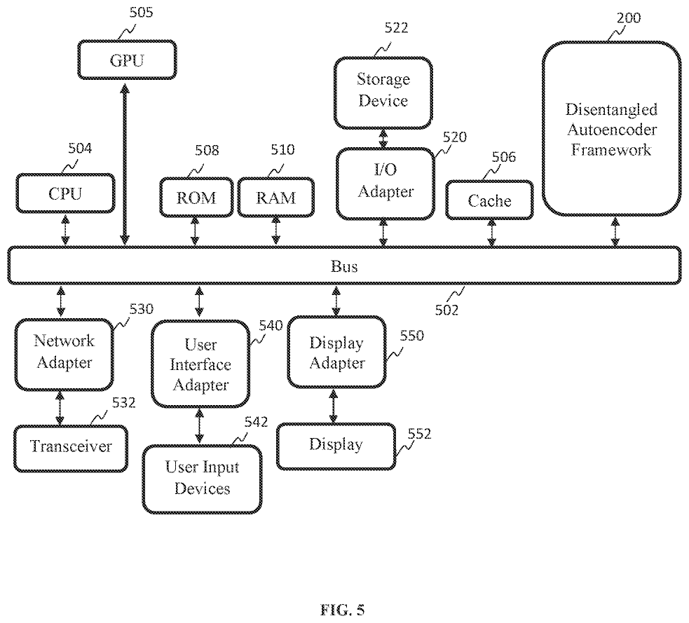

FIG. 5 is an exemplary processing system for employing a disentangled Wasserstein autoencoder for protein engineering, in accordance with embodiments of the present invention.

The processing system includes at least one processor (CPU) 504 operatively coupled to other components via a system bus 502. A GPU 505, a cache 506, a Read Only Memory (ROM) 508, a Random Access Memory (RAM) 510, an input/output (I/O) adapter 520, a network adapter 530, a user interface adapter 540, and a display adapter 550, are operatively coupled to the system bus 502. Additionally, a disentangled autoencoder framework 200 is connected to the bus 502 which aims to separate functional embeddings from structural embeddings.

A storage device 522 is operatively coupled to system bus 502 by the I/O adapter 520. The storage device 522 can be any of a disk storage device (e.g., a magnetic or optical disk storage device), a solid-state magnetic device, and so forth.

A transceiver 532 is operatively coupled to system bus 502 by network adapter 530.

User input devices 542 are operatively coupled to system bus 502 by user interface adapter 540. The user input devices 542 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 542 can be the same type of user input device or different types of user input devices. The user input devices 542 are used to input and output information to and from the processing system.

A display device 552 is operatively coupled to system bus 502 by display adapter 550.

Of course, the processing system may also include other elements (not shown), as readily contemplated by one of skill in the art, as well as omit certain elements. For example, various other input devices and/or output devices can be included in the system, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system are readily contemplated by one of ordinary skill in the art given the teachings of the present invention provided herein.

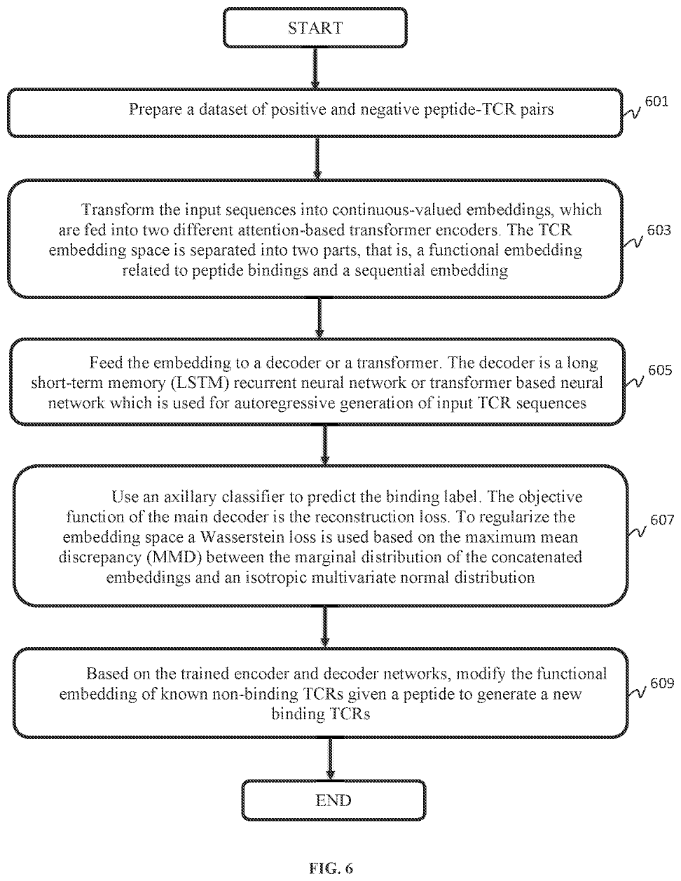

FIG. 6 is a block/flow diagram of an exemplary method for learning disentangled representations for employing a disentangled Wasserstein autoencoder for protein engineering, in accordance with embodiments of the present invention.

At block 601, prepare a dataset of positive and negative peptide-TCR pairs.

At block 603, transform the input sequences into continuous-valued embeddings, which are fed into two different attention-based transformer encoders. The TCR embedding space is separated into two parts, that is, a functional embedding related to peptide bindings and a sequential embedding.

At block 605, feed the embedding to a decoder. The decoder is a long short-term memory (LSTM) recurrent neural network or transformer based neural network which is used for autoregressive generation of input TCR sequences.

At block 607, use an axillary classifier to predict the binding label. The objective function of the main decoder is the reconstruction loss. To regularize the embedding space a Wasserstein loss is used based on the maximum mean discrepancy (MMD) between the marginal distribution of the concatenated embeddings and an isotropic multivariate normal distribution.

At block 609, based on the trained encoder and decoder networks, modify the functional embedding of known non-binding TCRs given a peptide to generate a new binding TCRs.

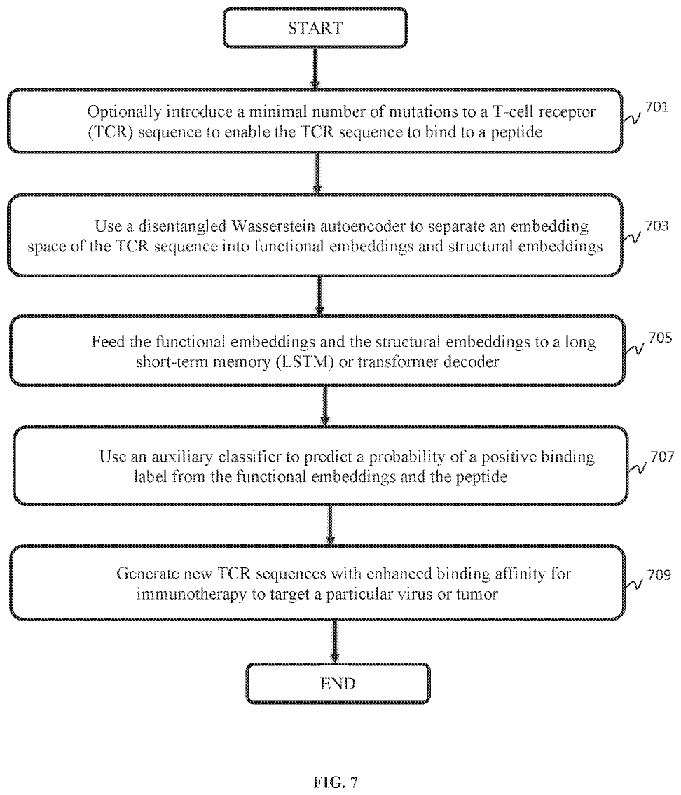

FIG. 7 is a block/flow diagram of an exemplary method for T-cell receptors to achieve precision immunotherapy, in accordance with embodiments of the present invention.

At block 701, optionally introduce a minimal number of mutations to a T-cell receptor (TCR) sequence to enable the TCR sequence to bind to a peptide.

At block 703, use a disentangled Wasserstein autoencoder to separate an embedding space of the TCR sequence into functional embeddings and structural embeddings.

At block 705, feed the functional embeddings and the structural embeddings to a long short-term memory (LSTM) or transformer decoder.

At block 707, use an auxiliary classifier to predict a probability of a positive binding label from the functional embeddings and the peptide.

At block 709, generate new TCR sequences with enhanced binding affinity for immunotherapy to target a particular virus or tumor.

Therefore, to automate this process from a data-driven perspective, the exemplary methods introduce a disentangled Wasserstein autoencoder with an auxiliary classifier, which isolates the function-related patterns from the rest with theoretical guarantees. This enables one-pass protein sequence editing and improves the understanding of the resulting sequences and editing actions involved. To demonstrate its effectiveness, it is applied it to T-cell receptors (TCRs), a well-studied structure-function case. It is shown that the exemplary method can be used to alter the function of TCRs without changing the structural backbone, outperforming several competing methods in generation quality and efficiency, and requiring only 10% of the running time needed by baseline models.

As used herein, the terms “data,” “content,” “information” and similar terms can be used interchangeably to refer to data capable of being captured, transmitted, received, displayed and/or stored in accordance with various example embodiments. Thus, use of any such terms should not be taken to limit the spirit and scope of the disclosure. Further, where a computing device is described herein to receive data from another computing device, the data can be received directly from the another computing device or can be received indirectly via one or more intermediary computing devices, such as, for example, one or more servers, relays, routers, network access points, base stations, and/or the like. Similarly, where a computing device is described herein to send data to another computing device, the data can be sent directly to the another computing device or can be sent indirectly via one or more intermediary computing devices, such as, for example, one or more servers, relays, routers, network access points, base stations, and/or the like.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” “calculator,” “device,” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical data storage device, a magnetic data storage device, or any suitable combination of the foregoing. In the context of this document, a computer readable storage medium may be any tangible medium that can include, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks or modules.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks or modules.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks or modules.

It is to be appreciated that the term “processor” as used herein is intended to include any processing device, such as, for example, one that includes a CPU (central processing unit) and/or other processing circuitry. It is also to be understood that the term “processor” may refer to more than one processing device and that various elements associated with a processing device may be shared by other processing devices.

The term “memory” as used herein is intended to include memory associated with a processor or CPU, such as, for example, RAM, ROM, a fixed memory device (e.g., hard drive), a removable memory device (e.g., diskette), flash memory, etc. Such memory may be considered a computer readable storage medium.

In addition, the phrase “input/output devices” or “I/O devices” as used herein is intended to include, for example, one or more input devices (e.g., keyboard, mouse, scanner, etc.) for entering data to the processing unit, and/or one or more output devices (e.g., speaker, display, printer, etc.) for presenting results associated with the processing unit.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for learning disentangled representations for T-cell receptors to improve immunotherapy, the method comprising:
optionally introducing a minimal number of mutations to a T-cell receptor (TCR) sequence to enable the TCR sequence to bind to a peptide;
using a disentangled Wasserstein autoencoder to separate an embedding space of the TCR sequence into functional embeddings and structural embeddings;
feeding the functional embeddings and the structural embeddings to a long short-term memory (LSTM) or transformer decoder;
using an auxiliary classifier to predict a probability of a positive binding label from the functional embeddings and the peptide; and
generating new TCR sequences with enhanced binding affinity for immunotherapy to target a particular virus or tumor.

2. The computer-implemented method of claim 1, wherein the functional embeddings include information about a generic sequential context and the structural embeddings encode patterns that are responsible for peptide recognition.

3. The computer-implemented method of claim 1, wherein a first and second auxiliary loss are employed to ensure the functional embeddings encode functional information while being independent of the structural embeddings.

4. The computer-implemented method of claim 3, wherein the first auxiliary loss is a Wasserstein loss based on a maximum mean discrepancy (MMD) between a marginal distribution of concatenated embeddings.

5. The computer-implemented method of claim 3, wherein the second auxiliary loss is an isotropic multivariate normal distribution loss.

6. The computer-implemented method of claim 1, wherein the functional embeddings correspond to functional patterns and the structural embeddings correspond to structural patterns.

7. The computer-implemented method of claim 1, wherein the functional embeddings are encoded by a functional encoder and the structural embeddings are encoded by a structural encoder.

8. A computer program product for learning disentangled representations for T-cell receptors to improve immunotherapy, the computer program product comprising a non-transitory computer readable storage medium having program instructions embodied therewith, the program instructions executable by a computer to cause the computer to perform a method comprising:
optionally introducing a minimal number of mutations to a T-cell receptor (TCR) sequence to enable the TCR sequence to bind to a peptide;
using a disentangled Wasserstein autoencoder to separate an embedding space of the TCR sequence into functional embeddings and structural embeddings;
feeding the functional embeddings and the structural embeddings to a long short-term memory (LSTM) or transformer decoder;
using an auxiliary classifier to predict a probability of a positive binding label from the functional embeddings and the peptide; and
generating new TCR sequences with enhanced binding affinity for immunotherapy to target a particular virus or tumor.

9. The computer program product of claim 8, wherein the functional embeddings include information about a generic sequential context and the structural embeddings encode patterns that are responsible for peptide recognition.

10. The computer program product of claim 8, wherein a first and second auxiliary loss are employed to ensure the functional embeddings encode functional information while being independent of the structural embeddings.

11. The computer program product of claim 10, wherein the first auxiliary loss is a Wasserstein loss based on a maximum mean discrepancy (MMD) between a marginal distribution of concatenated embeddings.

12. The computer program product of claim 10, wherein the second auxiliary loss is an isotropic multivariate normal distribution loss.

13. The computer program product of claim 8, wherein the functional embeddings correspond to functional patterns and the structural embeddings correspond to structural patterns.

14. The computer program product of claim 8, wherein the functional embeddings are encoded by a functional encoder and the structural embeddings are encoded by a structural encoder.

15. A computer processing system for learning disentangled representations for T-cell receptors to improve immunotherapy, comprising:
a memory device for storing program code; and
a processor device, operatively coupled to the memory device, for running the program code to:
optionally introduce a minimal number of mutations to a T-cell receptor (TCR) sequence to enable the TCR sequence to bind to a peptide;
use a disentangled Wasserstein autoencoder to separate an embedding space of the TCR sequence into functional embeddings and structural embeddings;
feed the functional embeddings and the structural embeddings to a long short-term memory (LSTM) or transformer decoder;
use an auxiliary classifier to predict a probability of a positive binding label from the functional embeddings and the peptide; and
generate new TCR sequences with enhanced binding affinity for immunotherapy to target a particular virus or tumor.

16. The computer processing system of claim 15, wherein the functional embeddings include information about a generic sequential context and the structural embeddings encode patterns that are responsible for peptide recognition.

17. The computer processing system of claim 15, wherein a first and second auxiliary loss are employed to ensure the functional embeddings encode functional information while being independent of the structural embeddings.

18. The computer processing system of claim 17, wherein the first auxiliary loss is a Wasserstein loss based on a maximum mean discrepancy (MMD) between a marginal distribution of concatenated embeddings.

19. The computer processing system of claim 17, wherein the second auxiliary loss is an isotropic multivariate normal distribution loss.

20. The computer processing system of claim 15,
wherein the functional embeddings correspond to functional patterns and the structural embeddings correspond to structural patterns; and
wherein the functional embeddings are encoded by a functional encoder and the structural embeddings are encoded by a structural encoder.
